(19) United States
(12) Patent Application Publication
(10) Pub. No.: US 2024/0358238 A1
(43) Pub. Date: Oct. 31, 2024

(54) SCOPE ADAPTERS FOR FLEXIBLE SCOPES

(71) Applicants: Boston Scientific Medical Device Limited, Galway (IE); Boston Scientific Scimed, Inc., Maple Grove, MN (US)

(72) Inventors: Steven T. CARLSON, St. Paul, MN (US); John SUBASIC, Stow, MA (US); Niraj Prasad RAUNIYAR, Plymouth, MN (US); Arun ADHIKARATH BALAN, Aluva (IN); Rajivkumar SINGH, Thane (IN)

(73) Assignees: Boston Scientific Medical Device Limited, Galway (IE); Boston Scientific Scimed, Inc., Maple Grove, MN (US)

(21) Appl. No.: 18/645,534

(22) Filed: Apr. 25, 2024

Related U.S. Application Data
(60) Provisional application No. 63/499,050, filed on Apr. 28, 2023.

(51) Int. Cl. A61B 1/00; A61B 1/01

(52) U.S. Cl. CPC A61B 1/00154; A61B 1/00066; A61B 1/00105; A61B 1/01

(57) ABSTRACT

A scope adapter system includes an adapter body coupled to a handle of a scope device, a rigid shaft, and a mounting mechanism removably coupled to the shaft. The shaft includes a shaft lumen which is sized and shaped to slidably receive a flexible elongated member of the device. A distal end of the lumen is sized so that, when a distal end of the member is received therein, the distal end of the member is snugly held so that an aim of the shaft aims the distal end of the member. The mechanism mounts a proximal end of the shaft to the body so that, when the body is coupled to the handle, the mechanism is mounted on the body and coupled to the shaft and the member is inserted into the lumen so that movement of the handle effects corresponding movements of the distal end of the member.

### CROSS-REFERENCE TO RELATED APPLICATIONS

This application claims the benefit of priority to U.S. Provisional Application No. 63/499,050, filed on Apr. 28, 2023, which is incorporated by reference herein in its entirety.

### TECHNICAL FIELD

The present disclosure relates to scope adapters and, in particular, to single use scope adapters which allow flexible scopes to perform the same functions as a rigid scope.

### BACKGROUND

During certain endoscopic procedures both rigid and flexible endoscopic devices may be used. For example, during kidney stone procedures a physician may use a rigid endoscopic device to navigate to the bladder, survey, and place guidewires before using a flexible endoscopic device to access the ureteral. However, endoscopic devices take time to set up and the supporting equipment takes up a significant amount of space in the operating room. Therefore, it is easier, cheaper, and requires less time to use fewer endoscopic devices during an endoscopic procedure. Thus, the present disclosure provides a scope adapter which allows a physician to use a flexible endoscopic device as they would use a rigid endoscopic device and thereby reducing the number of endoscopic devices used during certain procedures.

### SUMMARY

The present disclosure relates to a scope adapter system for guiding a flexible scope device toward a target area in a living body. The system includes an adapter body configured to be coupled to a handle of a scope device to be used with the scope adapter system. Also, the system includes a rigid shaft extending between a proximal end and a distal end. The rigid shaft includes a first shaft lumen extending therethrough. The first shaft lumen is sized and shaped to slidably receive a flexible first elongated member of the scope device therein. A distal end of the first shaft lumen is sized so that, when a distal end of the first elongated member is received therein, the distal end of the first elongated member is snugly held so that an aim of the rigid shaft aims the distal end of the first elongated member.

In addition, the system includes a mounting mechanism configured to be removably coupled to the rigid shaft. The mounting mechanism is configured to mount the proximal end of the rigid shaft to the adapter body so that, when the adapter body is coupled to the handle of the scope device, the mounting mechanism is mounted on the adapter body and coupled to the rigid shaft and the first elongated member is inserted into the first shaft lumen so that movement of the handle effects a corresponding movement of the distal end of the first elongated member.

In an embodiment, the mounting mechanism includes a first port which, when the mounting mechanism is coupled to the rigid shaft, is aligned with the first shaft lumen.

In an embodiment, the rigid shaft includes a second shaft lumen extending therethrough and wherein the mounting mechanism includes a second port aligned with the second shaft lumen, and the second shaft lumen and the second port are configured to receive a second elongated member therethrough.

In an embodiment, the scope adapter system further includes a distal tip extending distally from the distal end of the rigid shaft and including a first tip lumen open to the first shaft lumen, the first tip lumen being configured to receive the first elongated member therethrough and a second tip lumen open to the second shaft lumen to receive the second elongated member therethrough.

In an embodiment, the first shaft lumen includes a slot extending from the distal end of the first shaft lumen to the proximal end of the first shaft lumen, the slot being sized so that the first elongated member can be removed from the first shaft lumen laterally through the slot.

In an embodiment, the scope adapter system further includes a retention element selectively coupleable to the proximal end of the rigid shaft, wherein the retention element is configured so that, when coupled to the proximal end of the rigid shaft, movement of the first elongated member relative to the rigid shaft is prevented.

In an embodiment, the rigid shaft further includes a core member received within the rigid shaft to define the first shaft lumen, the core member being configured to snuggly receive the first elongated member therethrough.

In an embodiment, the rigid shaft is a metal shaft defining a first working channel configured to receive the flexible first elongated member therethrough and a second working channel configured to receive a second elongated member therethrough.

In an embodiment, the first and second shaft lumens are open to one another and wherein an opening between the first and second shaft lumens is sized to prevent the first elongated member from passing therethrough.

In an embodiment, the rigid shaft includes an outer member defining a single lumen and a core member inserted into the single lumen to divide the single lumen into the first and second shaft lumens.

In an embodiment, the scope adapter system further includes a coupling element at the distal end of the rigid shaft, the coupling element including a first tube that is sized and shaped to receive the rigid shaft therein and a second tube that is sized and shaped to receive the flexible first elongated member of the scope device therein.

In an embodiment, the flexible first elongated member is configured to be separated from the coupling element when a predetermined force is applied to remove the flexible first elongated member from the coupling element via the slot.

In an embodiment, the adapter body further comprises a handle configured to snap rigidly onto the scope device to permit a user to manipulate the rigid shaft.

In an embodiment, the adapter body includes a first body portion and a second body portion configured to be coupled together around the handle of the scope device to couple the adapter body to the handle of the scope device.

In an embodiment, the first and second body portions are configured to be coupled to one another via a plurality of securing features formed thereon.

In addition, the present disclosure relates to a method for treating tissue within a living body. The method includes attaching an adapter body to a handle of a flexible scope device; attaching the adapter body to a proximal end of a rigid shaft via a mounting mechanism including a first port aligned with a first shaft lumen of the rigid shaft; inserting a flexible shaft of the flexible scope device into the first shaft lumen, such that a distal end of the flexible shaft is proximal of a distal end of the rigid shaft; guiding the rigid shaft and the flexible shaft toward a target area within the living body; and manipulating the adapter body to aim the flexible scope device within the living body.

In an embodiment, the method further includes advancing the flexible shaft distally beyond a distal end of the rigid shaft; and operating a portion of the flexible shaft extending distally out of the rigid shaft using a steering system of the flexible scope device to access the target area within the living body.

In an embodiment, the method further includes separating the flexible shaft from the rigid shaft via a slot in the rigid shaft that is open to the first shaft lumen while the flexible shaft remains within the living body; and removing the rigid shaft from the living body while the flexible shaft remains within the living body.

In an embodiment, the mounting mechanism includes a second port aligned with a second shaft lumen of the rigid shaft. The method further includes inserting a second elongated member through the second port and the second shaft lumen; moving the second elongated member through the second shaft lumen toward the distal end of the rigid shaft; advancing the second elongated member distally out of the rigid shaft, such that a distal end of the second elongated member is distal of the distal end of the rigid shaft; and guiding the second elongated member toward the target area.

### BRIEF DESCRIPTION OF THE DRAWINGS

FIGS. 1A-1B show perspective views of a scope adapter according to an exemplary embodiment of the present disclosure.

FIGS. 2A-2D show perspective views of the distal end of a scope adapter according to exemplary embodiments of the present disclosure.

FIG. 3 shows a perspective view of a scope adapter according to an exemplary embodiment of the present disclosure.

FIGS. 4A-4C show perspective view of the distal end of a snap off scope adapter according to exemplary embodiments of the present disclosure.

FIGS. 5A-5B show a scope adapter according to another exemplary embodiment of the present disclosure.

FIGS. 6A-6B show a scope adapter according to yet another exemplary embodiment of the present disclosure.

### DETAILED DESCRIPTION

The present disclosure may be further understood with reference to the following description and the appended drawings, wherein like elements are referred to with the same reference numerals. The present disclosure relates to a scope adapter and, in particular, relates to a single use scope adapter for flexible scope devices, such as, cystoscopes and flexible nephroscopes. Exemplary embodiments of the present disclosure describe a scope adapter having a rigid shaft with at least one lumen allowing a flexible scope to advance down the adapter lumen and a body allowing the scope adapter to be attached to a scope device so that the flexible scope may perform substantially similar to a rigid scope. It should be noted that the terms “proximal” and “distal”, as used herein, are intended to refer to toward (proximal) and away from (distal) a user of the device.

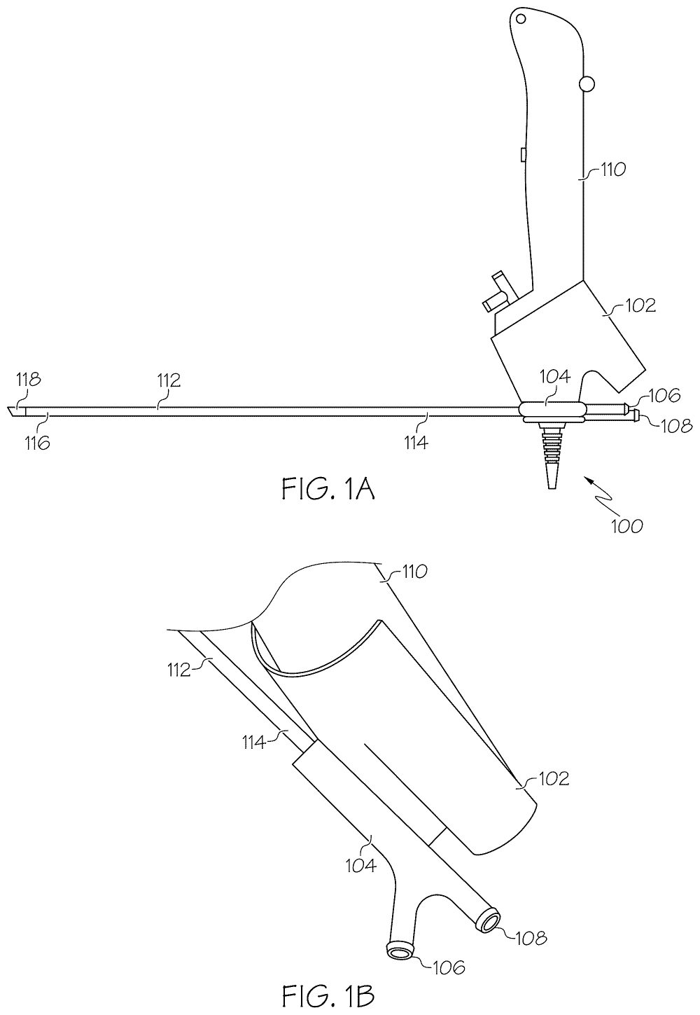

As shown in FIGS. 1A-1B, a scope adapter 100 according to an exemplary embodiment of the present disclosure comprises an adapter body 102 and a rigid shaft 112. The adapter body 102 is configured to be releasably attached to a scope device 110 (e.g., rigidly coupled), for example, an endoscope, ureteroscope, cystoscope, or nephroscope. In an exemplary embodiment, the scope device 110 is a LithoVue™ scope device. As those skilled in the art would understand, the adapter body 102 may have different shapes for use with different scopes, e.g., the adapter body 102 may be shaped to fit the corresponding scope device 110 so that the scope adapter 100 is ergonomically and releasably attachable to the scope device 110. The adapter body 102 additionally includes a mounting bracket 104 configured so that the rigid shaft 112 can be mounted onto the adapter body 102.

The mounting bracket 104 may include one or more ports so that a user may access the lumen(s) of the rigid shaft 112. In an exemplary embodiment the mounting bracket 104 has two ports 106, 108. In an exemplary embodiment the ports 106, 108 feature luer attachments or other features for attaching devices as known to those skilled in the art. As would be understood by those skilled in the art the mounting bracket 104 may be configured so that the rigid shaft 112 may be rigidly coupled to the adapter body 102. This permits a user to accurately move the rigid shaft 112 and a flexible shaft of the scope device 110 inserted into the rigid shaft 112 by manipulating the handle of the scope device 110 that is coupled to the adapter body 102.

The rigid shaft 112 extends from a proximal end 114 rigidly coupled to the mounting bracket 104 of the adapter body 102 to a distal end 116. The rigid shaft 112 may be releasably or permanently coupled to the mounting bracket 104. In an exemplary embodiment a distal tip 118 is located at the distal end 116 of the rigid shaft 112. The rigid shaft 112 includes at least one lumen extending from the distal end 116 to the proximal end 114 of the rigid shaft 112, the lumen including an opening at the proximal and distal ends 114, 116 of the rigid shaft 112. In an exemplary embodiment the rigid shaft 112 includes two lumens extending from the distal end 116 to the proximal end 114, both lumens being open at the proximal and distal ends 114, 116 of the rigid shaft 112.

In an exemplary embodiment the lumen(s) of the rigid shaft 112 are configured to align with the port(s) of the adapter body 102, so that a device (e.g., scope, imaging device, etc.) inserted into the port is directed to the correspondingly aligned lumen so that the device may be advanced through the port and down the length of the lumen to the distal end 116 of the rigid shaft 112. As would be understood by those skilled in the art the rigid shaft 112 may be any length. In an exemplary embodiment the rigid shaft 112 is twelve inches in length. In an exemplary embodiment the rigid shaft 112 is sized and shaped for insertion into a human urethra. As would be understood by those skilled in the art the rigid shaft 112 may be sized and shaped for insertion into any bodily orifice. Furthermore, as would be understood by those skilled in the art, at least the lumen which will receive the flexible scope may be curved to facilitate the insertion of the flexible scope.

As shown in FIGS. 1A-1B, a system according to an exemplary embodiment of the present disclosure comprises a scope adapter 100 configured to provide, when desired, rigid scope functionality to a flexible scope, such as the scope device 110. To provide rigid scope functionality to a flexible scope a user may rigidly attach the adapter body 102 to the scope device 110 (e.g., snap on, clip on, screw on, etc.). The user may rigidly attach the rigid shaft 112 to the adapter body 102 by inserting the proximal end 114 of the rigid shaft 112 into the mounting bracket 104.

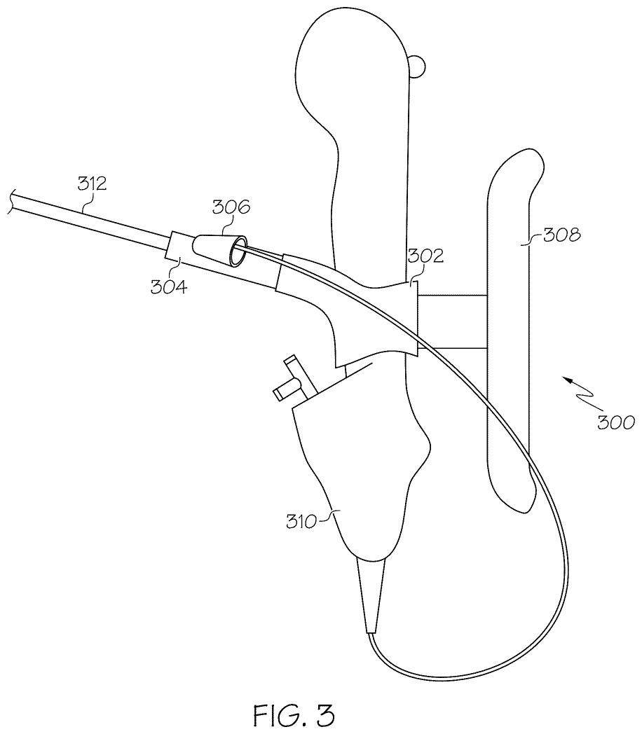

Alternatively, the rigid shaft 112 may be permanently attached to the adapter body 102. As would be understood by those skilled in the art, flexible scopes generally comprise an elongated member configured for insertion into a natural body lumen. In this configuration, the user may loop the elongated member of the scope device 110 or 310 so that the elongated member may be inserted into a port 106, 108, or 306 of the adapter body 102 or 302, as shown in FIG. 3. The elongated member of the scope device 110 may then be advanced to the distal tip 118 of the rigid shaft where the distal tip of the scope device 110 may remain. In this configuration the scope device 110 may be used as a rigid scope would be used. When rigid scope functionality is no longer desired, a user may remove the scope from the scope adapter 100 and use the scope device 110 as a flexible scope once more. To remove the scope adapter 100 from the scope device 110, the user may remove the elongated member from the rigid shaft 112 by moving the elongated member proximally down the rigid shaft 112.

Once the elongated member is removed from the rigid shaft 112, the user may detach the adapter body 102 from the scope device 110 (e.g., snap off, unclip, unscrew, etc.). The user may use the flexible scope in the normal manner. Alternatively, the scope adapter 100 may remain attached to the scope device 110 and the elongated member of the flexible scope may be advanced beyond the distal tip 118 of the rigid shaft 112 allowing the portion of the scope beyond the distal tip 118 to be used as a flexible scope.

As would be understood by those skilled in the art, during use the adapter body 102 remains outside of a living body while the rigid shaft 112 is configured to be inserted into the living body (via e.g., a naturally occurring bodily orifice) to provide access into a natural body lumen (such as the urethra or bladder) to which the rigid shaft 112 has been inserted. The rigid shaft 112 may be used in substantially the same manner as a rigid scope. The adapter body 102 includes two ports 106, 108 for accessing the lumens of the rigid shaft 112. The elongated member of a flexible scope may be inserted into either of the ports 106, 108 and advanced through a lumen of the rigid shaft 112 until a distal end of the elongated member is located at the distal tip 118 of the rigid shaft 112.

This may be done before or after the adapter 100 has been inserted to a target position in a body lumen. Another endoscopic device (e.g., a clipping device, a biopsy device, an imaging device, etc.) can then be inserted into the other port and advanced down the lumen of the rigid shaft 112 not occupied by the scope, though such an additional endoscopic device is not necessary for operation of the system. Those skilled in the art will understand that the scope may also include a working channel through which devices and/or fluids may be passed to the distal tip 118 of the rigid shaft 112.

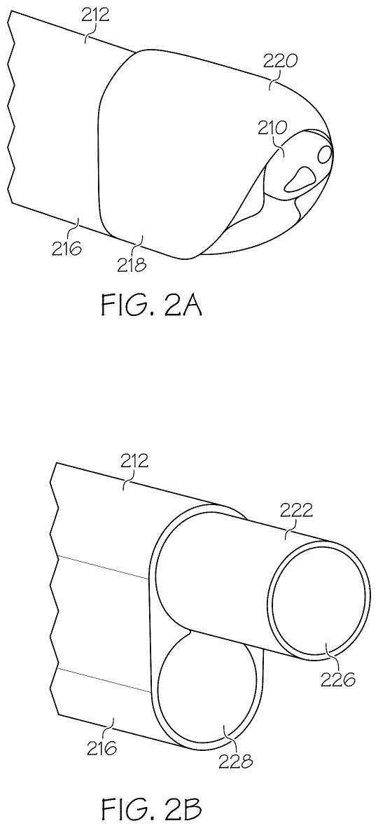

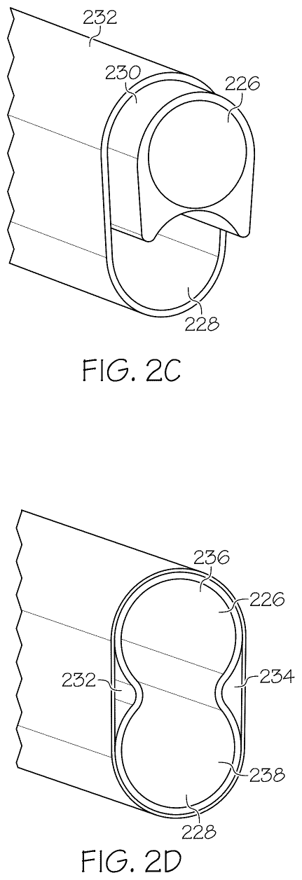

As shown in FIGS. 2A-2D, an exemplary embodiment includes a rigid shaft 212, a rigid shaft distal end 216, and a distal tip 218 distal of the rigid shaft distal end 216. In an exemplary embodiment the distal tip 218 has a shape (e.g., tapered) which makes anatomical navigation easier, such as a beak tip 220. However, as those skilled in the art will understand any desired tip shape may be used. In an exemplary embodiment the distal tip 218 is made from a bio compatible material, such as a molded polymer.

As those skilled in the art will understand the distal tip 218 may include lumen(s) open at proximal and distal ends of the distal tip 218 which align with the lumen(s) of the rigid shaft 212 to allow devices advanced down the lumen(s) of the rigid shaft 212 to advance down the lumen(s) of the distal tip 218 and access the bodily lumen beyond the distal tip 218. As those skilled in the art will understand the lumen(s) of the distal tip 218 will have the same width and shape as the lumen(s) of the rigid shaft 212, so that a lumen of the distal tip 218 aligned with a lumen of the rigid shaft 212 to define one continuous working channel.

The rigid shaft 212 includes at least one lumen extending through the entire length of the rigid shaft 212, from a proximal end (not shown) to the rigid shaft distal end 216. Each lumen of the rigid shaft 212 is open at each end of the rigid shaft 212 so that a device (e.g., a scope, clipping device, biopsy device, imaging device, etc.) can enter the lumen at a proximal end and be advanced through the rigid shaft 212 lumen until it reaches the rigid shaft distal end 216 of the lumen and exits the rigid shaft 212 lumen to enter the distal tip 218 lumen and/or enter the bodily lumen. In an exemplary embodiment a scope 210 is advanced down the lumen and positioned so that the distal end of the scope 210 remains in the distal tip 218 adjacent to the body lumen.

In this embodiment the scope functionalities (e.g., scope imagers, scope working channels, etc.) of the scope 210 may be used while the scope 210 and rigid shaft 212 are used as a rigid scope e.g., to take advantage of the superior accuracy in aiming that is often achievable with a rigid scope. In an exemplary embodiment the rigid shaft 212 has two lumens 226, 228. In an exemplary embodiment one of the lumens 226, 228 can be used for irrigation or suction removal of solids and liquids at the working site (e.g., drainage of the bladder) while the scope resides in the other lumen. The lumens 226, 228 may be equal in size or the lumens 226, 228 may be different sizes.

In an exemplary embodiment one of the rigid shaft 212 lumens will be sized and shaped to fit a scope 210 so that the scope 210 will be constrained from moving laterally and vertically, while being able to move proximally and distally. In an exemplary embodiment one of the rigid shaft 212 lumens is sized and shaped so that a scope 210 and a scope sheath may both be advanced down the rigid shaft 212 lumen. In an exemplary embodiment one of the rigid shaft lumens may be sized and shaped so that an endoscopic device may be advanced down the rigid shaft 212 lumen, the rigid shaft 212 lumen being sized and shaped so that the device is constrained from moving laterally and vertically, while being able to move proximally and distally, as this will ensure that the aiming of devices inserted through the rigid shaft 212 are aimed in accordance with the position and orientation of the rigid shaft 212.

The rigid shaft 212 may be made from any sufficiently biocompatible material, as would be understood by those skilled in the art. In an exemplary embodiment the rigid shaft 212 is a polymer extrusion and includes a metal core, such as steel core 222, to provide the rigid shaft 212 with a desired rigidity (e.g., rigidity sufficient to avoid significant deflection along its length when subjected to lateral forces by tissue adjacent to the body lumen in which the rigid shaft 212 is positioned). In this embodiment, the steel core 222 is a steel tube with a lumen 226 running the entire length of the steel core 222, the lumen 226 being open at both ends of the steel core 222. In another exemplary embodiment the rigid shaft 212 can be made entirely of metal, such as, steel. This steel shaft 232 may include an insert 230 (e.g., a polymer insert) to define the two lumens 226, 228. In an exemplary embodiment the insert 230 is made of a polymer.

However, as one skilled in the art would understand the insert may be made of any material sufficient to define the two lumen 226, 228 within a steel shaft 232. In an exemplary embodiment the steel core 222 or the insert 230 may extend from the proximal end of rigid shaft 212 to the rigid shaft distal end 216. In an exemplary embodiment (FIG. 2C), the steel core 222 or the insert 230 extends beyond the rigid shaft distal end 216. In this embodiment the steel core 222 or the insert 230 extends into the distal tip 218 to provide rigidity and/or define two working channels within the distal tip 218.

In another exemplary embodiment the steel shaft 232 has one lumen 226 and the steel shaft 232 is shaped to define two working channels 236, 238 within the lumen 226 (FIG. 2D). In this embodiment, a cross section of the steel shaft 232 has an oblong circular shape with an indent in each elongated side (e.g., a generally “figure-eight” shape) to define two substantially circular working channels 236, 238 within the lumen 226. The described steel shaft 232 lumen shape is exemplary and, as those skilled in the art will understand, the steel shaft 232 may be any shape which defines two working channels and the shape is designed to be atraumatic and to minimize a cross sectional area of the rigid shaft 212 while including lumens sized to accommodate the devices intended to be used therewith. In this embodiment the steel shaft 232 may include a cover 234 to give the rigid shaft 212 a more ergonomic shape. As those skilled in the art will understand the cover 234 may be applied through a heat shrink process or any appropriate process known in the art.

As shown in FIG. 3, a scope adapter 300 according to an exemplary embodiment of the present disclosure comprises an adapter body 302 and a rigid shaft 312. The adapter body 302 can be releasably attached to a scope device 310 creating a rigid coupling therebetween. The adapter body 302 comprises a mounting bracket 304 for rigidly mounting the rigid shaft 312 onto the adapter body 302 and the port 306 for accessing the lumen(s) of the rigid shaft 312. The adapter body 302 may also comprise a handle 308, rigidly attached to the adapter body 302, which allows the user to grip the scope adapter 300 for ergonomic use of the scope device 310 and scope adapter 300. The scope adapter 300 may be attached on any part of the scope device 310. The handle 308 of the scope adapter 300 may be located on any part of the adapter body 302.

In an exemplary embodiment the scope adapter 300 is attached on the middle portion of the scope device 310. In an exemplary embodiment the handle 308 is located directly proximal of the mounting bracket 304. In this embodiment the handle 308 is essentially in line with the rigid shaft 312 so that the user can maneuver the rigid shaft 312 easily and intuitively through manipulation of the handle 308. The handle 308 may be any shape. In an exemplary embodiment, handle 308 may be a bar attached to the adapter body 302 by a column or any other connecting structure located at the middle of the bar. One skilled in the art would understand that the connecting structure can connect to any portion of the handle 308 and can connect to any portion of the adapter body 302. In an exemplary embodiment the handle 308 is detachable from the adapter body 302.

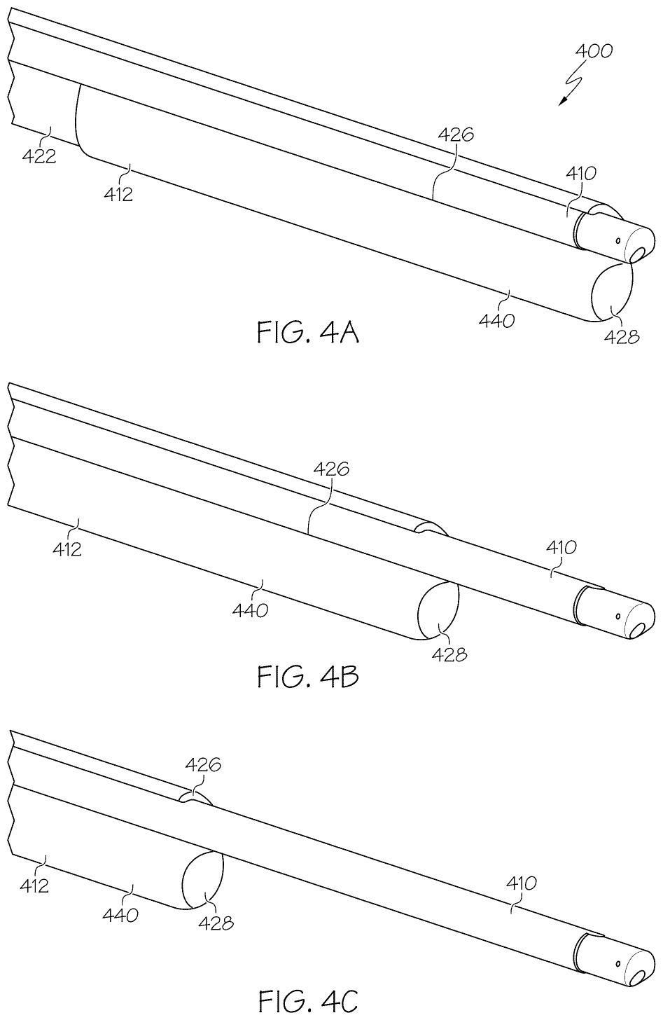

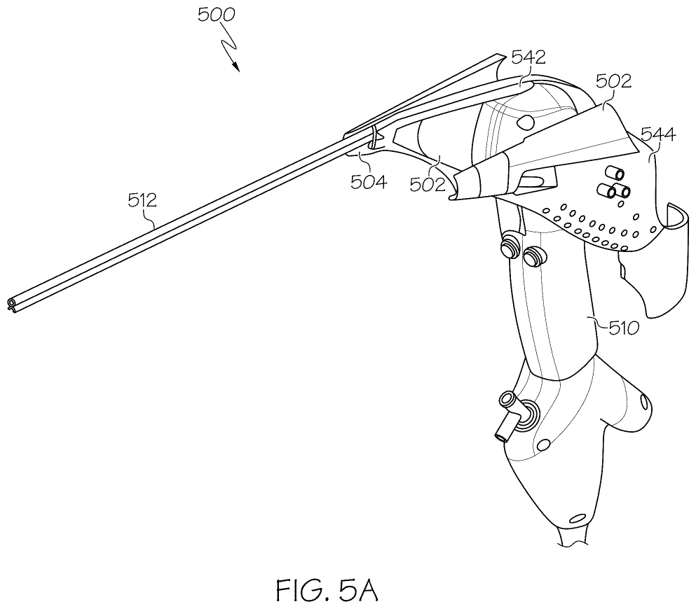

As shown in FIGS. 4A-4C, a scope adapter 400 according to an exemplary embodiment of the present disclosure includes a rigid shaft 412. In this exemplary embodiment the rigid shaft 412 comprises a steel core 422 and a snap element 440. In this exemplary embodiment the steel core 422 is a steel tube with a lumen running the entire length of the steel core 422, the lumen being open at both ends of the steel core 422. In this exemplary embodiment the steel core 422 is made from steel, however, as one skilled in the art would know any material may be used (e.g., other metals, certain polymers, etc.) to provide the rigid shaft 412 with a desired rigidity (e.g., rigidity sufficient to avoid significant deflection along its length when subjected to lateral forces by tissue adjacent to the body lumen in which the rigid shaft 412 is positioned). The snap element 440 may be located at the distal end of the rigid shaft 412. In an exemplary embodiment the snap element 440 has a lumen 428 and a slot 426. The lumen 428 is sized and shaped to receive the steel core 422 within the lumen. In an exemplary embodiment the steel core 422 may fit tightly in the lumen 428 so that the steel core 422 and the snap element 440 are rigidly attached.

As those skilled in the art will understand the steel core 422 may be permanently or releasably attached to the snap element 440. The slot 426 is sized and shaped to receive a flexible scope device 410 within the slot. In an exemplary embodiment the slot 426 may be sized and shaped so that the scope device 410 is constrained from moving laterally and vertically, while being able to move proximally and distally. In an exemplary embodiment the slot 426 may contain an opening in the lateral side sized and shaped so that the scope device 410 can be removed from the slot 426 of the snap element 440 through the lateral opening.

As shown in FIGS. 4A-4C, according to an exemplary embodiment of the present disclosure a user may attached the scope adapter 400 to the scope device 410 by inserting a distal end of the scope device 410 into a proximal end of the slot 426 and advancing the scope device 410 to a distal end of the slot 426. Alternatively, a user may insert the scope device 410 into the slot 426 through the lateral opening in the slot 426. In this exemplary embodiment a user would firmly press a distal portion of the elongated member of the scope device 410 into the lateral opening in the slot 426 until the scope device 410 snapped into the slot 426. Once the scope device 410 is in the slot 426 of the rigid shaft 412, the scope device 410 and scope adapter 400 may be inserted into the target bodily lumen and used in the manner of a rigid scope.

When the user wants to use the scope device 410 as a flexible scope, the user may leave the scope device 410 inside the bodily lumen and move the rigid shaft 412 proximally, thereby retracting the rigid shaft from the bodily lumen without retracting the scope device 410, as illustrated in FIG. 4C. Once the rigid shaft 412 is outside the bodily lumen, the user may remove the scope device 410 from the rigid shaft 412 by removing the scope device 410 from the slot 426 of the snap element 440 via the lateral opening in the slot 426. The user may remove the scope device 410 by peeling the snap element 440 off of the scope device 410. Once the scope device 410 has been removed from the rigid shaft 412 the scope device may be used as a flexible scope once more. This embodiment allows the user to switch the scope device 410 from rigid scope functionality to flexible scope functionality while leaving the scope inserted in the bodily lumen. By leaving the scope device 410 within the bodily lumen the user saves significant time as the user does not have to insert the scope device (or two separate scope devices) into the bodily lumen twice.

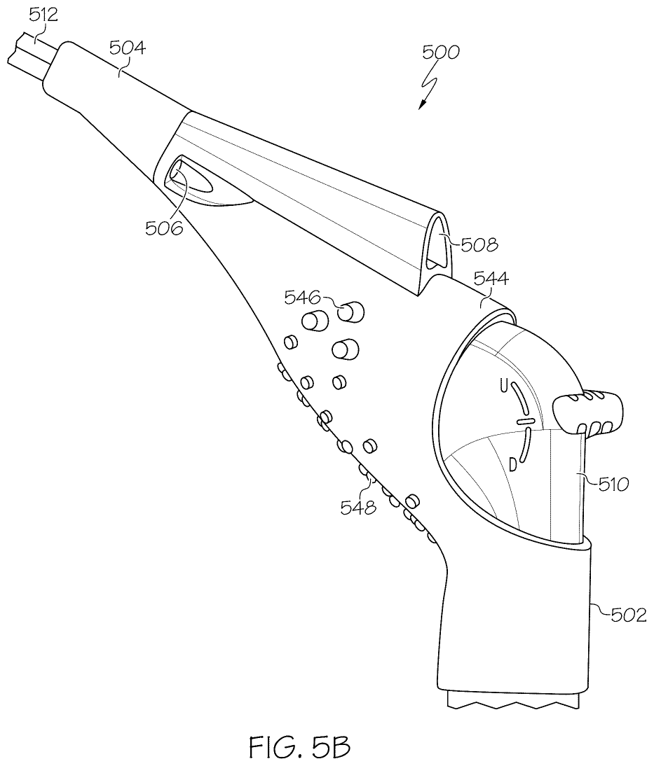

As shown in FIGS. 5A-5B, according to an exemplary embodiment of the present disclosure a scope adapter 500 includes an adapter body 502 and a rigid shaft 512. In an exemplary embodiment the adapter body 502 comprises two adapter body pieces 542, 544. The adapter body pieces 542, 544 are sized and shaped so that the adapter body pieces 542, 544 may be assembled, and later disassembled, around a scope device 510 to form the adapter body 502 rigidly coupled to the scope device 510. As those skilled in the art understand the adapter body 502 and adapter body pieces 542, 544 may have different shapes for use with different scopes, e.g., the adapter body 502 may be shaped to fit a corresponding scope device 510 so that the scope adapter 500 is ergonomically and releasably attachable to the corresponding scope device 510. In an exemplary embodiment the adapter body pieces 542, 544 may be substantially mirror images of each other.

As those skilled in the art understand the adapter body pieces 542, 544 may be different sizes and shapes from each other and may include different features from each other. In an exemplary embodiment the adapter body pieces 542, 544 may be press fit together and the adapter body pieces 542, 544 may include features to rigidly attach the adapter body pieces 542, 544 to each other and the scope device 510. In an exemplary embodiment the adapter body pieces 542, 544 may have pegs 546 for securing the adapter body pieces 542, 544 to the scope device 510. As those skilled in the art understand, the adapter body pieces 542, 544 may use other features for securing the adapter body pieces 542, 544 to the scope device 510, alone or in combination with pegs 546, such as adhesives, grooves, clips, press fittings, sockets, etcetera.

In an exemplary embodiment, when the adapter body 502 is assembled around the scope device 510 the adapter body may include two ports 506, 508 for accessing lumens of the rigid shaft 512 and a mounting bracket 504 for rigidly attaching the rigid shaft 512 to the adapter body 502. The adapter body 502 may include gripping features 548 to assist a user in gripping the scope adapter 500 and the scope device 510.

In an exemplary embodiment a user may attach the scope adapter 500 to the scope device 510 by placing the end of the rigid shaft 512 in the mounting bracket 504. A user may then place one of the adapter body pieces 542, 544 on the scope device 510 and then place the second one of the adapter body pieces 542, 544 on the scope device. The user may then press the two adapter body pieces 542, 544 together so that they form the adapter body 502. In an exemplary embodiment the adapter body pieces 542, 544 are snapped together and do not require any further securing. In an exemplary embodiment a user may secure the adapter body pieces 542, 544 to the scope device 510 and/or each other using the pegs 546 or other securing features.

As would be understood by those skilled in the art, flexible scopes generally comprise an elongated member configured for insertion into a natural body lumen. Once the user has attached the scope adapter 500 to the scope device 510 the user may loop the elongated member of the scope device 510 around and insert the elongated member into one of the ports 506, 508. Once inserted the elongated member of the scope device 510 may be advanced to the distal end of the rigid shaft 512 and the scope device 510 may be used as a rigid scope device would be used. When the scope adapter 500 is attached to the scope device 510, another endoscopic device (e.g., a clipping device, a biopsy device, an imaging device, etc.) may be inserted into the other port 506, 508 and advanced down the lumen of the rigid shaft 512 not occupied by the scope, though such an additional endoscopic device is not necessary for operation of the system.

When rigid scope functionality is no longer desired, a user may remove the scope from the scope adapter 500 and use the scope device 510 as a flexible scope once more. To remove the scope adapter 500 from the scope device 510, the user may remove the elongated member from the rigid shaft 512 by moving the elongated member proximally down the rigid shaft 512. Once the elongated member is removed from the rigid shaft 512, the user may dissemble the adapter body 502 by unattaching the adapter body pieces 542, 544 from each other and the scope device 510. Once disassembled the scope device 510 may be used as a flexible scope once more.

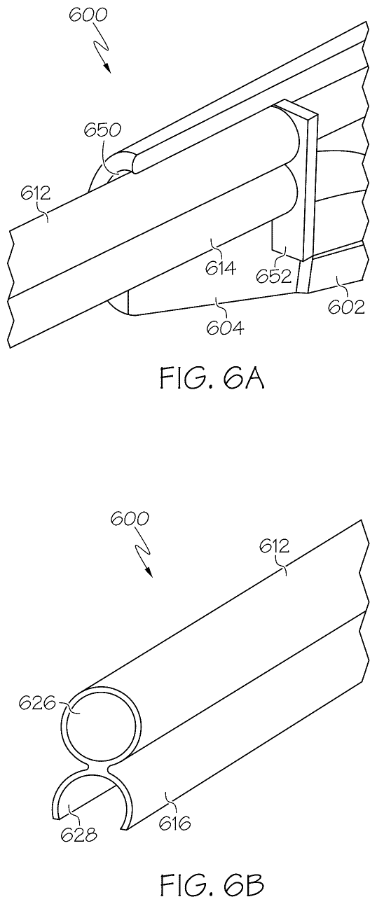

As shown in FIGS. 6A-6B, a scope adapter 600 according to an exemplary embodiment of the present disclosure includes a rigid shaft 612 and an adapter body 602. In an exemplary embodiment the adapter body 602 has a mounting bracket 604 which includes a socket 650. The socket 650 is sized and shaped to house the proximal end 614 of the rigid shaft 612. In an exemplary embodiment the socket 650 may have a shape that corresponds to an imprint of the proximal end 614 of the rigid shaft 612. In an exemplary embodiment the adapter body 602 may comprise two adapter body pieces (such as those shown in FIG. 5A), in this embodiment each adapter body piece may have a socket 650 which is configured to house the proximal end 614 of the rigid shaft 612 when the adapter body pieces are coupled together. In this exemplary embodiment the proximal end 614 of the rigid shaft 612 is housed within the socket 650 so that the rigid shaft 612 is rigidly coupled to the adapter body 602 and the rigid shaft 612 will not move independently of the adapter body 602.

In an exemplary embodiment, the rigid shaft 612 may have a retention element 652 at the proximal end 614, sized and shaped so that when the retention element 652 is mounted in socket 650 the rigid shaft 612 is unable to move. In an exemplary embodiment the rigid shaft 612 retention element 652 may be a rectangular shape. As those skilled in the art would understand the retention element 652 may be any shape (e.g., circular, square, triangular, irregular, etc.) that effectively prevents the rigid shaft 612 from moving independent of the adapter body 602 when the rigid shaft 612 is housed in the socket 650.

In an exemplary embodiment the rigid shaft 612 extends from a distal end 616 to a proximal end 614. The rigid shaft 612 may include a lumen 626 extending through a length of the rigid shaft 612 and open at the proximal and distal ends 614, 616 of the rigid shaft 612. The rigid shaft 612 may include a slot 628 extending the entire length of the rigid shaft 612 and open at the proximal and distal ends 614, 616 of the rigid shaft 612. The slot 628 may be substantially tubular with a portion of the tube wall open to the exterior of the tube. In an exemplary embodiment the slot 628 may be sized and shaped so that the elongated member of a flexible scope device can be inserted into the slot 628 and moved proximally and distally through the slot 628 while being constrained from moving laterally and vertically.

What is claimed is:

1. A scope adapter system for guiding a flexible scope device toward a target area in a living body, comprising:
an adapter body configured to be coupled to a handle of a scope device to be used with the scope adapter system;
a rigid shaft extending between a proximal end and a distal end, the rigid shaft including a first shaft lumen extending therethrough, the first shaft lumen being sized and shaped to slidably receive a flexible first elongated member of the scope device therein, a distal end of the first shaft lumen being sized so that, when a distal end of the first elongated member is received therein, the distal end of the first elongated member is snugly held so that an aim of the rigid shaft aims the distal end of the first elongated member; and
a mounting mechanism configured to be removably coupled to the rigid shaft, the mounting mechanism being configured to mount the proximal end of the rigid shaft to the adapter body so that, when the adapter body is coupled to the handle of the scope device, the mounting mechanism is mounted on the adapter body and coupled to the rigid shaft and the first elongated member is inserted into the first shaft lumen so that movement of the handle effects a corresponding movement of the distal end of the first elongated member.

2. The scope adapter system of claim 1, wherein the mounting mechanism includes a first port which, when the mounting mechanism is coupled to the rigid shaft, is aligned with the first shaft lumen.

3. The scope adapter system of claim 2, wherein the rigid shaft includes a second shaft lumen extending therethrough and wherein the mounting mechanism includes a second port aligned with the second shaft lumen, and the second shaft lumen and the second port are configured to receive a second elongated member therethrough.

4. The scope adapter system of claim 3, further comprising:
a distal tip extending distally from the distal end of the rigid shaft and including a first tip lumen open to the first shaft lumen, the first tip lumen being configured to receive the first elongated member therethrough and a second tip lumen open to the second shaft lumen to receive the second elongated member therethrough.

5. The scope adapter system of claim 1, wherein the first shaft lumen includes a slot extending from the distal end of the first shaft lumen to the proximal end of the first shaft lumen, the slot being sized so that the first elongated member can be removed from the first shaft lumen laterally through the slot.

6. The scope adapter system of claim 1, further comprising:
a retention element selectively coupleable to the proximal end of the rigid shaft, wherein the retention element is configured so that, when coupled to the proximal end of the rigid shaft, movement of the first elongated member relative to the rigid shaft is prevented.

7. The scope adapter system of claim 1, wherein the rigid shaft further comprises a core member received within the rigid shaft to define the first shaft lumen, the core member being configured to snuggly receive the first elongated member therethrough.

8. The scope adapter system of claim 1, wherein the rigid shaft is a metal shaft defining a first working channel configured to receive the flexible first elongated member therethrough and a second working channel configured to receive a second elongated member therethrough.

9. The scope adapter system of claim 3, wherein the first and second shaft lumens are open to one another and wherein an opening between the first and second shaft lumens is sized to prevent the first elongated member from passing therethrough.

10. The scope adapter system of claim 9, wherein the rigid shaft includes an outer member defining a single lumen and a core member inserted into the single lumen to divide the single lumen into the first and second shaft lumens.

11. The scope adapter system of claim 5, further comprising:
a coupling element at the distal end of the rigid shaft, the coupling element including a first tube that is sized and shaped to receive the rigid shaft therein and a second tube that is sized and shaped to receive the flexible first elongated member of the scope device therein.

12. The scope adapter system of claim 11, wherein the flexible first elongated member is configured to be separated from the coupling element when a predetermined force is applied to remove the flexible first elongated member from the coupling element via the slot.

13. The scope adapter system of claim 1, wherein the adapter body further comprises a handle configured to snap rigidly onto the scope device to permit a user to manipulate the rigid shaft.

14. The scope adapter system of claim 1, wherein the adapter body includes a first body portion and a second body portion configured to be coupled together around the handle of the scope device to couple the adapter body to the handle of the scope device.

15. The scope adapter system of claim 14, wherein the first and second body portions are configured to be coupled to one another via a plurality of securing features formed thereon.

16. A method for treating tissue within a living body, comprising:
attaching an adapter body to a handle of a flexible scope device;
attaching the adapter body to a proximal end of a rigid shaft via a mounting mechanism including a first port aligned with a first shaft lumen of the rigid shaft;
inserting a flexible shaft of the flexible scope device into the first shaft lumen, such that a distal end of the flexible shaft is proximal of a distal end of the rigid shaft;
guiding the rigid shaft and the flexible shaft toward a target area within the living body; and
manipulating the adapter body to aim the flexible scope device within the living body.

17. The method of claim 16, further comprising:
advancing the flexible shaft distally beyond a distal end of the rigid shaft; and
operating a portion of the flexible shaft extending distally out of the rigid shaft using a steering system of the flexible scope device to access the target area within the living body.

18. The method of claim 17, further comprising:
separating the flexible shaft from the rigid shaft via a slot in the rigid shaft that is open to the first shaft lumen while the flexible shaft remains within the living body; and
removing the rigid shaft from the living body while the flexible shaft remains within the living body.

19. The method of claim 16, wherein the mounting mechanism includes a second port aligned with a second shaft lumen of the rigid shaft, further comprising:
inserting a second elongated member through the second port and the second shaft lumen;
moving the second elongated member through the second shaft lumen toward the distal end of the rigid shaft;
advancing the second elongated member distally out of the rigid shaft, such that a distal end of the second elongated member is distal of the distal end of the rigid shaft; and
guiding the second elongated member toward the target area.
